(19) United States
(12) Patent Application Publication
(10) Pub. No.: US 2012/0143046 A1
(43) Pub. Date: Jun. 7, 2012

(54) ELECTRO-CHEMICAL-DEPOSITION OF GALFENOL AND THE USES THEROF

(76) Inventors: Bethanie J.H. Stadler, Shoreview, MN (US); Kotha Sai Madhukar Reddy, Minneapolis, MN (US); Douglas A. Rekenthaler, Woodbine, MD (US)

(21) Appl. No.: 13/309,673

(22) Filed: Dec. 2, 2011

Related U.S. Application Data
(60) Provisional application No. 61/419,667, filed on Dec. 3, 2010.

(51) Int. Cl. A61B 5/05; H02N 2/18; A61M 37/00; C25D 3/56

(52) U.S. Cl. 600/424; 205/238; 310/26; 604/20

(57) ABSTRACT

A method for the electro-chemical-deposition (ECD) of alloys of iron (Fe) and gallium (Ga) to electro-deposit magnetostrictive “Galfenol” thin films. Various uses and applications for said Galfenol thin films are also disclosed.

### CROSS-REFERENCE TO RELATED APPLICATION

This application claims the benefit of U.S. Provisional Application No. 61/419,667, filed Dec. 3, 2010, the entire disclosure of which is incorporated by reference herein.

### FIELD OF THE INVENTION

Embodiments described herein relate generally to methods and apparatuses for electro-depositing an alloy of iron (Fe) and Gallium (Ga), also known as “Galfenol,” as a thin film on a broad spectrum of possible substrates, and uses thereof.

### BACKGROUND

Galfenol (Fe₁₋ₓGaₓ where x=10-40%) is a magnetostrictive material that has promise to revolutionize active micro- and nano-electromechanical systems (MEMS/NEMS). Bimorph structures with magnetostrictive thick films have been used in MEMS devices as actuators and for energy harvesting. Many such applications require tensile integrity, ductility, long fatigue lifetimes and large magnetostriction constants. However, most materials that exhibit large magnetostriction, like Terfenol-D (˜1600 ppm), are far from possessing the mechanical qualities required for many applications due to having a tensile strength under 30 MPa and very brittle natures. Shape memory alloys, such as Ni₂MnGa, require difficult crystalline order to preclude martensitic variants from cancelling each other's response.

The Fe₁₋ₓGaₓ alloy system (Galfenol), was discovered in 1999 and has been studied in bulk form by several groups. The magnetostrictive response of Galfenol is linear, occurs in low fields (<150 Oe), and can be processed to eliminate the need for a biasing compressive stress. While its low coercivity, H_{c}, means less energy loss magnetically, it can lead to conductivity losses in the bulk form due to eddy currents.

However, bulk lamination minimizes the conductivity loss, and in films the effect would be negligible with appropriate geometric design. The cubic magnetic anisotropy of Galfenol varies between 3-7×10⁴ J/m³ reaching a maximum of 6.5×10⁴ J/m³ at 5 at % Ga. Although these alloys have been found to have more modest magnetostriction than Terfenol-D, up to 400 ppm, their mechanical properties, including tensile strengths greater than 440 MPa, and very ductile behavior, enable a new class of applications for magnetostrictives. Other geometries for Galfenol include melt-spun ribbons 15-100 μm thick, sputtered films, and nanowires.

Most magnetostrictive materials require components that are difficult to grow via aqueous electrochemical deposition (ECD), which is the traditional method for fabricating thick films (2-100 μm). Galfenol is no exception, with a desired Ga content of 10-40%. Thus, there exists a need for methods and apparatuses in order to enable Galfenol growth using electrochemical deposition.

### SUMMARY

The present invention is the first successful, comprehensive demonstration which overcomes the electrochemical challenges required to electroplate Galfenol. Combinatorial electrochemical deposition methods have been used to study complexing agents, bath composition, and overpotential in order to enable Galfenol growth, thereby opening an inexpensive path to the integration of magnetostrictive actuators, transducers, and strain, torque, and acoustic sensors. Using the electrochemical deposition tools developed here, Galfenol nanostructures, including rods, cones, cylinders, and spheres, can also be grown into nanoporous templates. This invention allows the use of fixed or dynamic masks that can alter the growth patterns, and thus the configuration of the electro-deposited devices. Batch processing for a single object can be accomplished, or a system can be set up to allow continuous processing, for example by moving ribbons, tapes, or surfaces through a deposition chamber.

### BRIEF DESCRIPTION OF THE DRAWINGS

FIG. 1 shows a Scanning Electron Microscope (SEM) image of a mixture of FeGa and FeGaO phases obtained during thin film electro-deposition. The insets of FIG. 1 show magnified images of the two regions.

FIG. 2 shows a cyclic voltammetry curve obtained for FeGa electro-deposition.

FIGS. 3a-3d are charts of the oxygen incorporation into the electro-deposit as a function of applied potential and solution pH for various solutions disclosed herein (e.g., in Table 1).

FIG. 4a shows a crystal of FeGa alloy during the initial stages of electro-deposition. FIG. 4b shows a thin film of pure FeGa phase with 76% Fe and 24% Ga.

FIG. 5 is a flowchart of an example method disclosed herein.

FIGS. 6-14 illustrate example devices incorporating galfenol thin films in accordance with embodiments disclosed herein.

### DETAILED DESCRIPTION

A method for the electro-chemical-deposition (ECD) of alloys of iron (Fe) and gallium (Ga) may be used to electro-deposit magnetostrictive “Galfenol” thin films. Galfenol has the unique features of being magnetostrictive, auxetic, ductile, and machinable. In thin film form, it has potential for use in sensors, actuators, and energy harvesting applications, among others. In development of the electrochemical deposition, four parameter spaces have been identified in which Galfenol and iron-gallium oxideswere produced. The inventors have shown that a broad range of overpotentials could be used to produce Galfenol of desired composition by varying the concentration of Ga³⁺ and its complexing agent, sodium citrate, compared to that of Fe²⁺.

Although iron alloy deposition has been studied for several decades, the present invention is the first successful electrochemical deposition of high-Ga content GaFe alloys, which is nontrivial via aqueous baths for the following reasons. The Pourbaix diagram for Ga shows that there are only a few voltage-pH combinations in which Ga³⁺ remains stable in aqueous solutions. When the pH exceeds 2.56 or 3.20, Ga³⁺ forms GaOH²⁺ or GaO⁺, respectively, in the absence of a complexing agent for Ga³⁺. When attempting to reduce the Ga³⁺ ions to the Ga state, the potential must be below the point at which hydrogen ions begin to reduce. Otherwise, without a pH buffer, a local depletion of H⁺ ions at the cathode causes an increase in pH which results in iron-gallium oxide formation. Electrochemical deposition of Galfenol alloys has been achieved with compositional and microstructural control.

Galfenol alloys, electro-plated on various substrates have now been shown to be very promising as functional materials in magnetostriction-based sensors, actuators, and energy harvesters. While bulk processing of the alloys has been substantially developed in the last decade since the discovery of Galfenol, thin films of FeGa alloys have only seen limited studies using vapor and electrochemical deposition. In contrast to vacuum deposition techniques, electro-deposition allows a fast and cost-effective route of processing such alloys, and it opens the possibilities of conformal coating of complex parts, such as the recesses of an intricately designed MEMS cavity.

Electro-deposition of FeGa alloys has been investigated earlier. For example, U.S. Pat. No. 7,834,490, which is hereby incorporated by reference in its entirety, discloses a method for electro-plating of a nonlinear bi-metallic energy harvesting device, comprised of Galfenol and an Aluminum substrate, wherein the bimetallic material is electro-plate. However, the complex interplay between various parameters (pH, overpotential, concentration) was never investigated thoroughly, and the complex problems were left unsolved until this present invention. The inventors of the present invention disclosed herein present experimental data backed by a phenomenological model that explains the reasons behind such complexities as generally observed during electrochemical deposition of FeGa alloys.

Pt sputtered Si wafers have been used as working electrodes. Prior to use, the electrodes may be cleaned with HCl followed by thorough washing with DI water. The counter electrode may be a large Pt sheet. Potentials are measured against a Ag/AgCl reference electrode. Electrolyte solutions are prepared from analytical grade chemicals (Alfa-Aesar). The pH is adjusted using NaOH. Cyclic Voltammetry and Chronoamperometry studies are conducted using Gamry Potentiostat. A JEOL-6500 SEM and EDS is used to examine the film morphology and composition after electro-deposition.

During electro-deposition of FeGa alloys as thin films, SEM-EDS analysis shows that the electrodeposits obtained are a mixture of two phases, one a pure FeGa phase, and the other a mixed FeGa-oxide phase. The extent of each phase is observed to be dependent on the experimental conditions used (the potential applied, solution pH, and electrolyte concentration). FIG. 1 is a representative SEM image obtained for one such experimental-parameter combination. The insets 101, 102 of FIG. 1 show the morphology of each phase. To the naked eye, a pure FeGa phase appears bright and shiny, a probable reason for which is revealed by brightly lit grains in the top right inset 101 comprised solely of the FeGa phase. The grains can be seen to be larger and shinier than the ones in FeGaO phase shown in the top left inset 102. The composition of the FeGaO phase of inset 102 is 54% Fe, 10% Ga, and 36% O. The composition of the FeGa phase of inset 101 is 76% Fe and 24% Ga.

While the ratio of Fe:Ga in the FeGa phase is 76% Fe+24% Ga, that within the FeGa-oxide phase is 84%:16% indicating that the oxygen presence in FeGaO phase is not due to oxidation, but rather due to its incorporation during the electro-deposition itself. This further points to inhibition of Ga deposition when there is a simultaneous O deposition. In order to understand this phenomenon, a detailed investigation shows the effects of each possible contributing factor. Furthermore, since practical thin film electro-deposition is sought on large sized electrodes, optimal conditions for electro-deposition of pure FeGa alloys on such electrodes have also been successfully investigated.

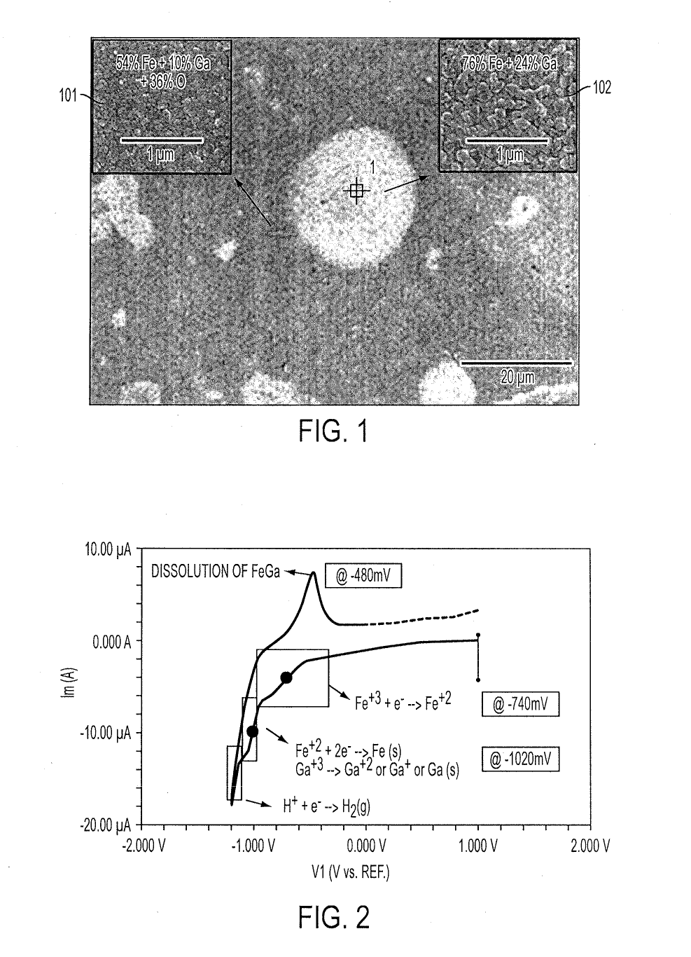

FIG. 2 shows a cyclic voltammetry curve obtained for FeGa electro-deposition. Here, the potential was ramped between +1 V and −1.2 V at a rate of 500 mV/s. For potentials more negative than −1 V (that is, towards the left), deposition of FeGa alloy occurs with a concomitant hydrogen evolution reaction. Over-potential for hydrogen evolution is pH dependent, and furthermore its occurrence affects the morphology and constituents of the deposited phase.

TABLE 1
Various electrolytes used in the course of the development
of the present invention

| | [Fe + 2] | [Ga + 3] | [Citrate-] |
|---|---|---|---|
| Electrolyte | (moles/cm3) | (moles/cm3) | (moles/cm3) |
| A | 0.015 | 0.025 | 0.015 |
| B | 0.030 | 0.025 | 0.015 |
| C | 0.015 | 0.050 | 0.015 |
| D | 0.015 | 0.025 | 0.030 |

Table 1 above shows the various electrolytes used in the course of developing the present invention. One constituent in each of the solutions B, C and D was made twice as concentrated as that in solution A. For instance, solution B has twice the amount of Fe+2 than solution A. This allows one to quantify the effect of each constituent on the composition of the electro-deposit obtained.

Additionally, for each of the above solutions, the effect of applied potential was investigated between −1090 mV and −1200 mV, the potential range in which cyclic voltammetry (FIG. 200) indicated a metal deposition reaction. Furthermore, the effect of bulk pH between 3.0 and 5.0 was also investigated, the pH being varied by addition of NaOH. Chronoamperometry studies were conducted following which the samples were analyzed for morphology and composition by SEM-EDS studies.

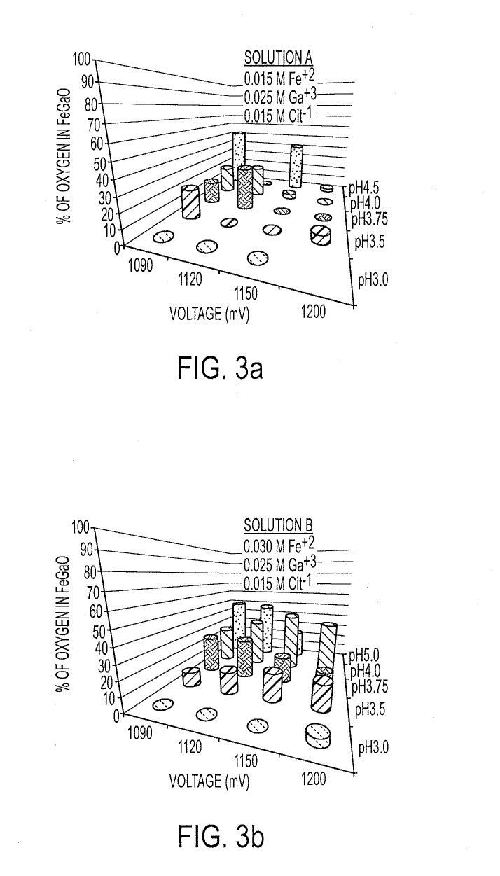

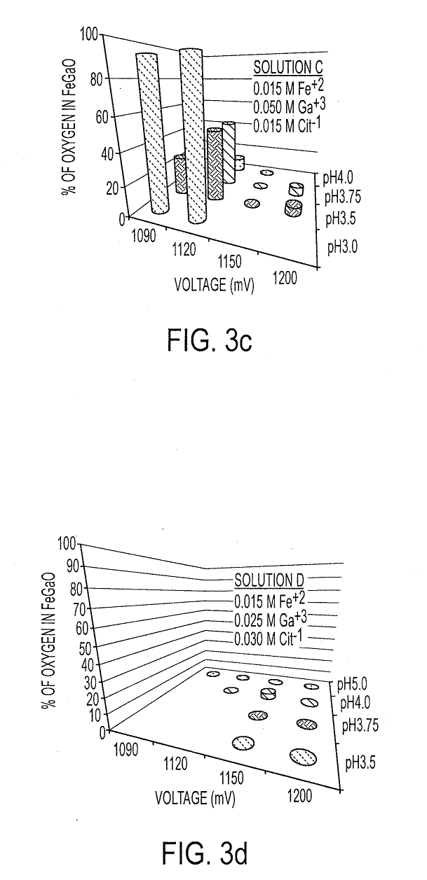

FIGS. 3a-3d summarize the results obtained from the chronoamperometry studies, and the preferred implementation of the electrochemical deposition process. As seen in FIG. 1, the electrodeposits obtained were a mixture of pure FeGa and mixed FeGa-oxide phases. FIGS. 3a-3d summarize how the oxygen incorporation into the electro-deposit varies as a function of applied potential and solution pH for various solutions used in Table 1. Wherever the amount of metallic alloy deposited was statistically insignificant compared to the Pt substrate, the data points are left blank, while pure FeGa alloy with zero oxygen atomic percentage is shown with zero length height.

Two problems have been resolved with respect to the disclosed and novel electrochemical deposition process. First, is the presence of hydrogen evolution side reactions. The extent of these reactions increases from moderate to high from Solution A to C, but is absent when the citrate concentration is increased as in Solution D. Secondly, the extent of oxygen incorporation in the electro-deposit correlates with the occurrence of hydrogen evolution reactions.

Also, the parameter space for oxygen incorporation is different for different solutions. Solution A results in oxygen incorporation at low applied potentials and high pH values. For solution B, the O incorporation occurs everywhere except at low pH values. For solution C, low applied potentials result in O incorporation into the electrodeposits. On the contrary, for solution D, practically no O incorporation is observed at any of the pH values and potentials used.

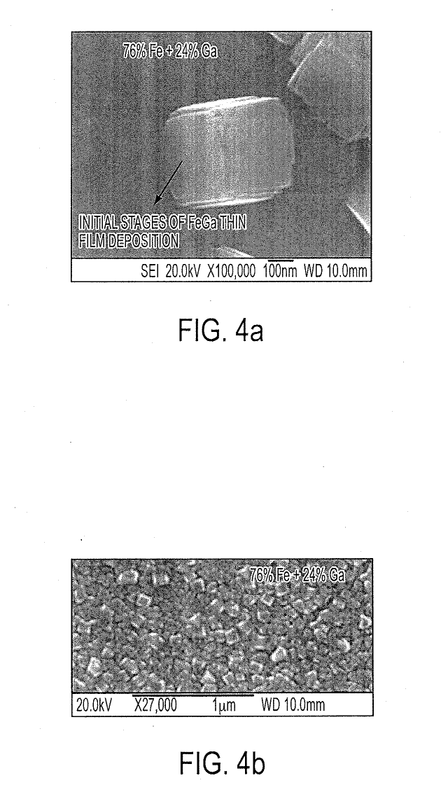

FIG. 4a shows a crystal of FeGa alloy during the initial stages of electro-deposition. One can notice a layer-by-layer growth of the FeGa crystal. FIG. 4b shows a thin film of pure FeGa phase with 76% Fe and 24% Ga.

A salient feature of the above results is the correlation between the FeGa-oxide phase formation and concomitant hydrogen evolution reaction. This correlation admits of two explanations: the hydrogen evolution reaction causes FeGa-oxide phase formation, or the hydrogen evolution reaction is caused by FeGa-oxide phase formation. In the first scenario, the pH rise caused near the cathode by hydrogen evolution reaction stabilizes the gallium hydroxide phases, which then reduce to lower valence gallium oxide phases like GaO and Ga2O. In the second scenario, metallic Ga(s) formed by the reduction of Ga+3 ions re-oxidize partially to lower valence gallium oxide phases upon coming in contact with water, while releasing hydrogen, shown by the following reaction (or more completely by reactions 5A and 6A below):

Ga(s)+H₂O→GaO or Ga₂O+H₂ (not balanced)

It is found that high pH conditions precipitate scenario 1 while low pH conditions precipitate scenario 2. Thus a phenomenological model has been built that successfully explains FeGa electro-deposition. This model is inspired by a similar model for induced co-deposition of transition metal-Molybdenum alloys.

### The Phenomenological Model

It is assumed that the iron group metal reduction occurs in two steps via an adsorbed intermediate. The reactions' equations are shown below:

[Fe(II)HCit]⁻²+e⁻→[Fe(I)HCit]⁻²_{ads} (1A)

[Fe(I)HCit]⁻²_{ads}+e⁻→Fe(s)+HCit⁻³ (2A)

This idea that a monovalent iron intermediate ion adsorbs at the electrode surface has been used to describe the anomalous co-deposition of NiFe alloys, where the preferentially adsorbed iron species blocks Ni reduction reaction. In the present model, the intermediate iron species affects gallium reduction similarly, by competing for available surface sites on the electrode. Furthermore, the gallium reduction is catalyzed by the adsorbed Fe⁺² species (see Eq. 3A). Equation 4A shows the reduction of Ga(III) species to FeGa(s) metal, which as shown in reactions 5A and 6A may re-oxidize to lower valence iron-gallium oxides.

Complexation of Ga+3 ion and subsequent reduction to Ga metal:

Ga(III)+[Fe(I)HCit]⁻²_{ads}→[Fe(I)Ga(III)HCit]⁺_{ads} (3A)

[Fe(I)Ga(III)HCit]⁺_{ads}+ne→Fe—Ga(s) (4A)

Formation of FeGa oxides by reaction with water at low pH (scenario 2):

Fe—Ga(s)+H₂O→Fe—GaO(s)+H₂ (5A)

2Fe—Ga(s)+H₂O→Fe—Ga₂O(s)+H₂ (6A)

Formation of FeGa oxides due to scenario 1:

H₂O+e⁻→½H₂+OH⁻ (1B)

Ga(III)+3OH⁻→Ga(OH)₃ (2B)

Ga(OH)₃+e⁻→Ga₂O or GaO (not balanced) (3B)

As discussed above, the effect that the hydrogen evolution reaction (1B) has on the overall FeGa electro-deposition is to stabilize the intermediate hydroxide (2B) resulting in lower valency gallium oxide phases (3B).

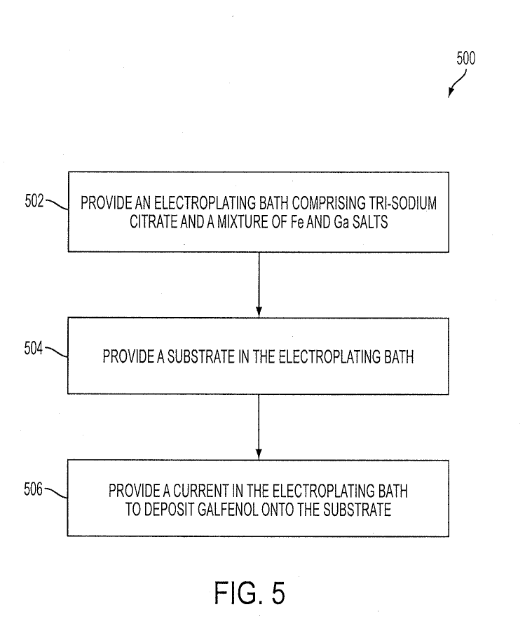

Referring now to FIG. 5, a method 500 of electro-plating FeGa (Galfenol) alloys, also termed electro-chemical-deposition, is now described. The method 500 comprises providing an electroplating bath comprising tri-sodium citrate and a mixture of Fe and Ga salts (step 502); providing a substrate in the electroplating bath (step 504); and providing a current in the electroplating bath to deposit Galfenol onto the substrate (step 506). The Galfenol product may include iron (Fe) and Gallium (Ga) in ratios which may vary from about 65% to 95% Fe, and about 5% to 35% Ga. In various embodiments, the Galfenol may include other trace elements at percentages of about 0% to 5% of the plated material. The Galfenol may be electro-chemically deposited onto various substrates, also called host surfaces or materials.

The electro-chemical-deposition bath used to deposit the Galfanol may include water, sodium citrate, sodium hydroxide, and a mixture of Fe and Ga salts, for example Fe sulfate and Ga sulfate. Fe sulfate and Ga sulfate are used to supply Fe²⁺ and Ga³⁺, respectively. Sodium citrate is used to complex the Ga³⁺ and Fe⁺² while sodium hydroxide is used to adjust the pH.

In one embodiment, the Fe²⁺:Ga³⁺ ratios may be between about 1:3-1:2 in the electro-chemical-deposition bath. In another embodiment, the Fe²⁺:Ga³⁺ ratio is about or exactly 3:7, or 0.015M Fe²⁺ to 0.035M Ga³⁺. The sodium citrate level may be equal to or less than that of Ga⁺³, for example, 0.035M sodium citrate. The pH may be adjusted to induce preference for film growth over the competing H₂ evolution reaction. In various embodiments, the pH values may be between about 3-6, but may vary depending on the bath composition.

A cathode made of platinum or other inert metals known in the art is arranged in the electroplating bath. In various embodiments, the Galfenol is electro-chemically deposited at carefully controlled temperatures, pressures, concentrations, and electric currents. In various embodiments, the temperature may be between 1° C. to about room temperature and ambient pressures and fields may be used. The processing environment may include controlled radio-frequency and electro-magnetic ambient fields, with a static or dynamic reactive plating chamber.

The deposition voltages are evaluated using cyclic voltammetry (CV) and chronoamperometry (CA) for each bath. The cyclic voltammetry defines the reduction potential range. Any voltage more negative than the galfenol deposition potential (see FIG. 2) will result in galfenol deposition. For the chronoamperometry, the voltage is fixed and the metal deposition versus hydrogen evolution is monitored by adjusting the pH. The same procedure may be repeated for baths of varying composition. In one embodiment, the galfenol deposition voltage range may be from −1.09 to −1.2V with respect to an Ag/AgCl reference.

In one embodiment, the following guidelines may be followed to achieve successful electro-plating of Galfenol. Microscopic hydrogen bubble evolution must be prevented in the process. However, such bubbles cannot be detected with the naked eye. In one embodiment, an anodic aluminum oxide (AAO) substrate may be used to indirectly discern gas evolution because the pores will be blocked and no current detected. The formation of bubbles may be prevented by adjusting the pH accordingly as mentioned above.

In another embodiment, the microscopic bubbles may be detected by growing the bubbles long enough to have them coalesce so that they may be seen, or by growing the bubbles in nanopores. After deposition, the nanoporous matrix may be etched and bubble evolution during deposition may be inferred from the unfilled pores that previously contained the bubbles. The pH of the bath may be adjusted accordingly to prevent bubbles in the next process.

The ratio of Fe⁺² to Ga⁺³ ions in the solution should be very small due to anomalous adsorption of Fe⁺² on the electrode. There is an optimum amount of citrate for a given Fe⁺² and Ga⁺³ concentration in the solution. Too little citrate will result in iron-gallium oxide precipitation. Too much citrate will allow reduction of citrate to form hydrogen gas. The pH and overpotential of the solution may be adjusted to match a narrow window in which FeGa of desired composition can be deposited repeatedly. At too high a pH or low overpotentials, there are low Ga content films produced. At too low a pH or high overpotentials, there is low Ga content films and/or hydrogen evolution. In various embodiments, a Cyclic Voltammetry with a one-millimeter-diameter inert ‘disk’ may be used to analyze the potential range for Galfenol deposition, and stripping for each of the above combinations.

Batch processing modes may be used to electro-plate the Galfenol. In other embodiments, a continuous electro-plating processing mode may be employed to produce strips, ribbons, or sheets. The method may be used for the electro-plating of 2-dimensional and/or 3-dimensional rods, cones, spheres, plates cylinders, blocks, for filling voids of various dimensions, for use in macro- NEMS- and MEMS-scale products.

In one embodiment, a static mask may be used to electro-plate patterns, grids, linear arrays, or complex 2-dimensional or 3-dimensional products. In another embodiment, the mask may be dynamic, or multiple masks may be used to create products with enclosed volumes. Multiple layers of Galfenol, or multiple layers of Galfenol and other substrates may be included on a single product. A Galfenol-plated product may have additional metal alloys of a different type plated on the surface of the Galfenol layup.

The Galfenol may be plated in “cells” in a geometric line array or grid. Vibrational, mechanical, thermal, radio-frequency, or electro-magnetic field impulses are impingent on, or manifested on the Galfenol cells and the effects of those impulses or fields are sensed and measured by the anisotropic response of the Galfenol material. The Galfenol cell response is sensed by the magnetic response of the Galfenol cell. The electro-plated Galfenol may be deposited on integrated circuits or other electronic devices to act as sensor, actuator, or energy source.

Multiple layers of Galfenol and/or other substrates and intermediate materials may be electro-deposited to attain specific responses from the combination of materials, such as piezo-electric materials bonded with Galfenol to augment energy production.

A thin layer of Galfenol on a substrate may capture surface wave effects which can be discriminated by sensing the anisotropic response of the Galfenol surface, grid, or linear array.

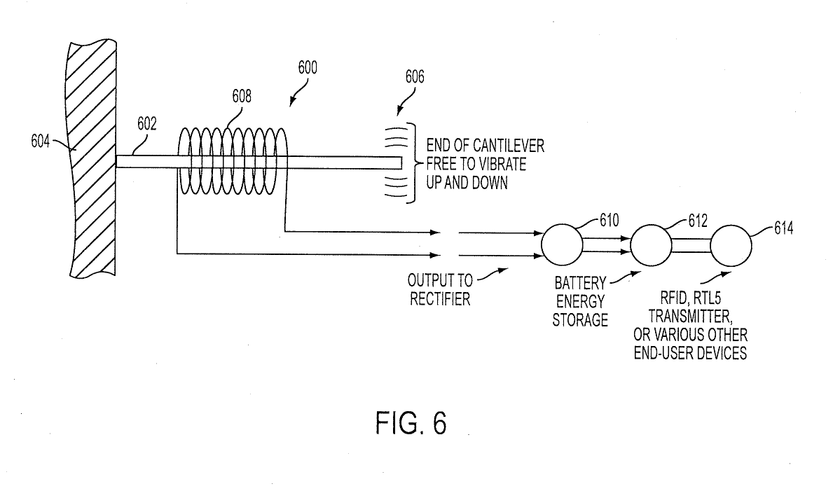

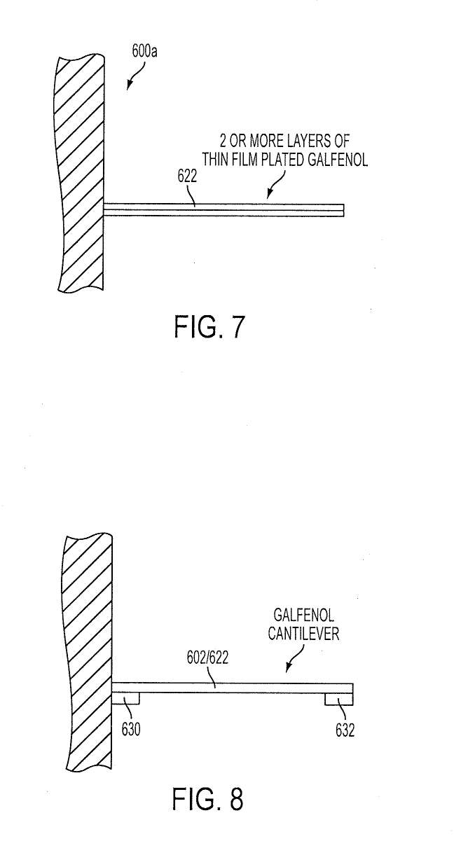

Referring now to FIG. 6, a device 600 for generating power from a vibrating cantilever, fabricated from brass, aluminum, or other material with a thin coating of Galfenol is now disclosed. The device 600 comprises a layer of Galfenol-plated, thin strip cantilever 602, fixed at one end by a fixture 604 and free to vibrate up and down at the other end 606. FIG. 7 illustrates an alternate device 600a comprising a cantilever 622 consisting of multiple Galfenol-plated, thin strip layers, where the strips are bonded using an adhesive.

One or more coils 608 of magnet wire is wound around the Galfenol strip(s), either in contact with the strip(s), or with small spacing between the coil and the strip(s) to allow free motion of the cantilever 602 (or 622). It should be appreciated that FIG. 7 (as well as FIGS. 8-10) does not illustrate any coils for clarity purposes. The motion of the cantilever 602 (or 622) induces a current through the coil due to the Faraday effect. The output from the cantilever-induced current in the coil 608 is fed to a rectifier 610. The output of the rectifier 610 is fed to a storage battery 612 for use in a device 614 such as e.g., RFID, RTL5, transmitters, communications circuits/devices or other devices.

In an alternate configuration, the Galfenol strip(s) may be replaced by Galfenol wires. In another configuration, the strips or wires may be of varying lengths, so as to change the harmonic frequency of the oscillations, and thus the output bandwidth of the device 600, 600a. In yet another configuration, the Galfenol cantilever may be bent at varying angles so as to pre-stress the material to maximize current generation.

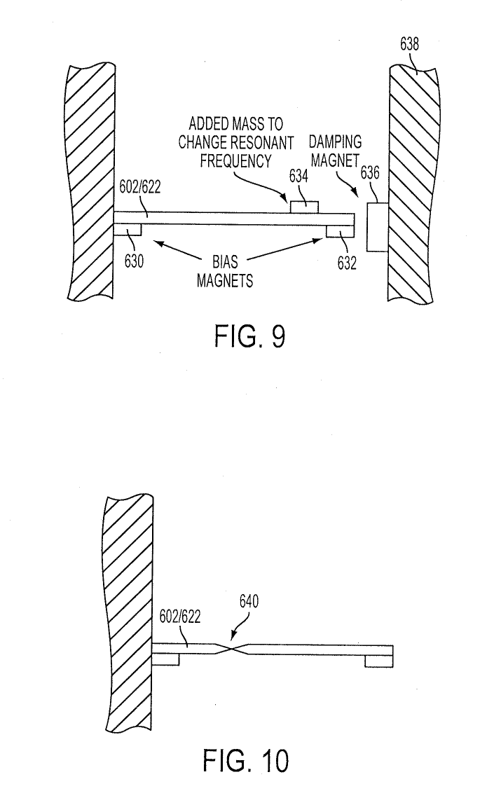

As shown in FIG. 8, the Galfenol strip(s) 602/622 may have bias magnets 630, 632 attached to both ends of the strips 602/622 so as to induce greater power output. In another embodiment, the Galfenol cantilever may have a mass 634 (FIG. 9) attached to its free end so as to modify the harmonic vibration frequency.

As shown in FIG. 9, in yet another configuration, a third magnet 636—a damping magnet—may be mounted close to (via another fixture 638), but not touching, the free end of the cantilever 602/622, so as to interact with the bias magnet on that end of the cantilever 602/622 with a resulting damping of the cantilever motion, and thus its frequency.

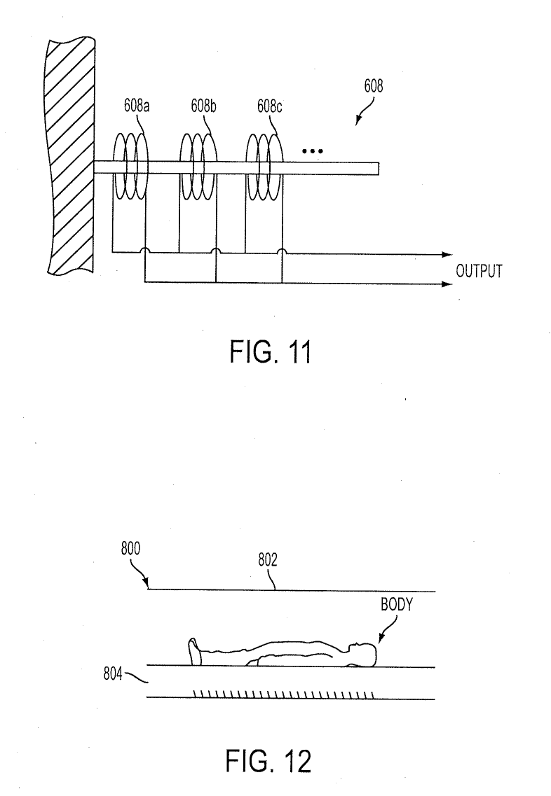

As shown in FIG. 10, the cantilever 602/622 may be twisted 90 degrees (at 640), such that vibrations in two lateral directions will stress the Galfenol and induce current flow. As shown in FIG. 11, the magnetic coil wire 608 may be wound in sections 608a, 608b, 608c, etc. in “salami-slice” style, so that the cantilever 602/622 can be twisted or curled. In another configuration, the magnet wire may be a bifilar winding.

Various combinations of the above features may be employed for various applications to maximize bandwidth and to select the appropriate harmonic frequency for particular uses. A typical embodiment will use most, if not all of these optional configurations in a single device.

In particular, a Galfenol-based power generator may be installed in conditions, comprising rotating, vibrating machinery, in fluid ocean, river, or lake wave motion, or at locations with available wind-driven waves to capture and harvest random sources of energy from anthropomorphic or naturally-occurring sources of vibratory motion.

In another embodiment, Galfenol may be used in a method of preventing injurious bodily joint motions by sensing longitudinal movement of an orthotic bandage, using the auxetic expansion of Galfenol fibers, yarns, ribbons, tapes, or plated fibers, yarns, ribbons, tapes to tighten and prevent motion beyond certain pre-determined limits in adverse directions.

Referring now to FIG. 12, an internal body imaging device 800 that exploits the inverse Galfenol magnetostrictive effect is now described. The device 800 may perform a method of internal body imaging by the exploitation of the inverse Galfenol magnetostrictive effect, whereby an external radio-frequency, electric, or magnetic field causes deflection of a Galfenol nanowire or nanostructure—the inverse Galfenol effect—wherein a remote field can cause deflection of the Galfenol nanowire. That deflection may be observed from above by a variety of means, including optical sensors to detect the relative motion and position of the nanowire tips, or at the base of the nanowires using sensors such as Giant Magnetic Resistors (GMRs)—techniques familiar to the magnetic hard drive and recording industries.

In one such embodiment, a linear array of Galfenol nanowires 804, which may be stationary or moving, above or below the body whose internal structures are to be imaged, with RF, electrical, or magnetic (B) field source 802 in opposition (below or above the body), such that the field effects are modulated by the intervening body, and variations in the body structure will be manifested by variations in deflection of the nanowires. In one configuration, the deflections may be sensed using optical sensors viewing the tops of the nanowires. In another configuration, the deflections and the correlated changes in their current output may be sensed using giant magnetic resistance sensors operating near the base of the nanowires. In another configuration, the linear array may be replaced by a grid matrix in two dimensions, if desired. In another configuration, the array 804 may be stationary and the RF, electrical, or magnetic field source 802 will move. In another configuration, the array 804 may be in motion and the RF, electrical, or magnetic field source 802 is stationary.

In all configurations, the internal structure of the body may be investigated in detail using methods familiar to those experienced in the art of image generation such as the Radon, Hough, Fourier, and myriad other transforms;

In yet another embodiment, radio-frequency (RF) and/or magnetic (B) fields may be passed through an object or body intervening between the source of the RF or B fields and a Galfenol grid or linear array which is used for sensing the RF and B fields, for the purpose of forming a high-resolution image of the interior of the intervening object.

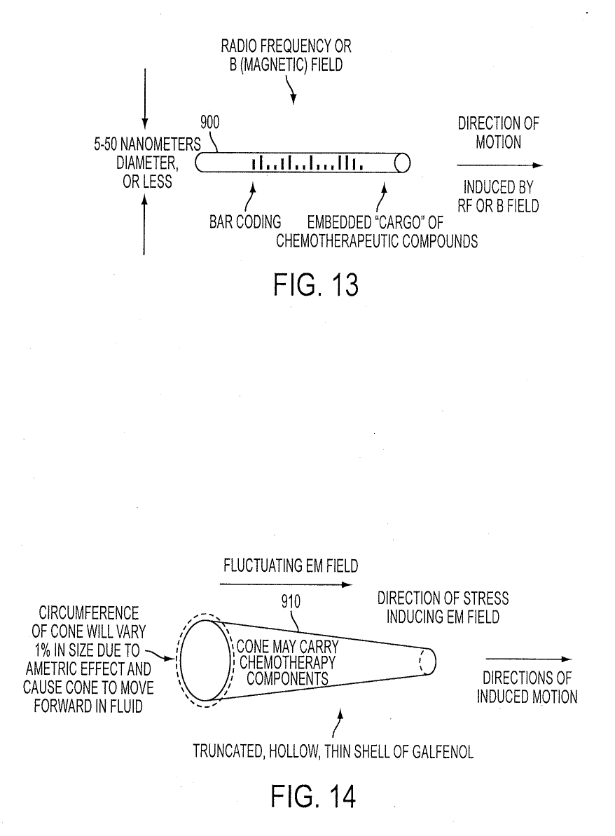

FIG. 13 illustrates an ECD fabrication of a Galfenol-plated nanowire(s) 900 used for vivo human and animal medical diagnostics and therapeutics. FIG. 13 illustrates a nanowire 900, but is should be appreciated that the same principles described below apply to nanoparticles, nanobots (“bot” being used here to mean “robot”) and other nanostructures. The nanowire 900, etc. comprises galfenol nanoparticles with embedded, attached chemotherapeutic compounds (the “cargo”), comprising molecules, viruses, proteins, enzymes, cells, chemicals, whereby the nanoparticle, nanowire, or nanostructure (“transporter”) may be injected, and subsequently controlled, monitored, tracked, and/or guided to a desired target point in the body for sensing, therapy, or to be triggered within the body by external radio-frequency (“RF”) or magnetic fields.

In one embodiment, Galfenol nanowires of varying diameter and length maybe fabricated, such that an external RF or magnetic field may induce heating of the nanowire, with the objective of thermal interaction with the targeted cell or body feature.

In another embodiment, nanobots of structural shape such that the auxetic feature of the Galfenol causes a synchronous change in the dimensions and shape of the structure, resulting in a propulsion of the structure through fluids such as might be found throughout the body. In one such configuration, the nanobot may take the form of a hollow-truncated cone 910 (FIG. 14), such that a magnetic stress in one direction will induce an expansion and volumetric change in a second dimension, inducing a “swimming” impulse, forcing the cone along the direction of the magnetic stress.

In another configuration, pulsating radio-frequency emissions may be used to induce corresponding pulsations in the cone's 910 diameter, resulting in a longitudinal vibration of the nanobot, with potential mechanical effects on the surrounding tissues and cells, to include the possible destruction of cell walls, selective resection, or ablation of cell constituents.

In another configuration, the nanobots may be bar-coded, enabling remote readout of the location and effectiveness of a particular cargo, as transported on an individual nanobot. Using large numbers of selectively coded magnetic nanobots simultaneously, enables discrimination of the relative effects of diverse therapies.

The above description and drawings are only to be considered illustrative of specific embodiments, which achieve the features and advantages described herein. Modifications and substitutions for specific conditions and materials can be made. Accordingly, the embodiments are not considered as being limited by the foregoing description and drawings, but is only limited by the scope of the appended claims.

What is claimed as new and desired to be protected by Letters Patent of the United States is:

1. A method of electro-plating a Galfenol alloy onto a substrate, comprising:
providing an electroplating bath comprising tri-sodium citrate and a mixture of Fe and Ga salts;
providing a substrate in the electroplating bath; and
providing a current in the electroplating bath to deposit Galfenol onto the substrate;
wherein the Fe²:Ga³⁺ ratio is between about 1:3-1:2, the amount of sodium citrate is equal to or less than that of Ga³⁺, and the pH is between about 3-6 in the electroplating bath.

2. A device for generating power from a vibrating cantilever, fabricated from brass, aluminum, or other material with a thin coating of Galfenol, the device comprising:
one of more layers of Galfenol-plated, thin strips, fixed at one end, free to vibrate up and down at another end; and
one or more coils of magnet wire wound around the one or more Galfenol strips, either in contact with the strips, or with small spacing between the coil and the strips to allow free motion of the cantilever, said motion inducing a current through the one or more coils due to the Faraday effect;
a rectifier for inputting the output from the cantilever-induced current in the one or more coils; and
a storage battery for inputting an output of the rectifier.

3. The device of claim 2, wherein the stored output is used in a sensor or in a communications circuit.

4. The device of claim 2, wherein the cantilever is twisted 90 degrees, such that vibrations in two lateral directions will stress the Galfenol and induce current flow.

5. The device of claim 2, wherein the one or more Galfenol strips are Galfenol wires.

6. The device of claim 5, wherein the Galfenol wires are of varying lengths, so as to change the harmonic frequency of output oscillations and output bandwidth.

7. The device of claim 2, wherein the cantilever may be bent at varying angles so as to pre-stress the material to maximize current generation.

8. The device of claim 2 wherein the one or more Galfenol strips may have bias magnets attached at both ends so as to induce greater power output.

9. The device of claim 8, wherein the cantilever may have a mass attached to the free end, so as to modify harmonic vibration frequency.

10. The device of claim 8, wherein a third magnet may be mounted close to, but not touching, the free end of the cantilever, so as to interact with the bias magnet on that end of the cantilever with a resulting damping of the cantilever motion and its frequency.

11. The device of claim 2, wherein the one or more coil is a bifilar winding.

12. The device of claim 11, wherein the one or more coils are wound in sections so that the cantilever can be twisted or curled.

13. A device for internal body imaging by exploitation of the inverse Galfenol magnetostrictive effect, whereby an external radio-frequency, electric, or magnetic field causes deflection of a Galfenol nanowire or nanostructure, wherein a remote field causes deflection of the Galfenol nanowire, the deflection may be observed from above by a variety of means, including optical sensors to detect the relative motion and position of the nanowire tips, or at the base of the nanowires using sensors such as Giant Magnetic Resistors (GMRs).

14. The device of claim 13, further comprising:
a linear array of Galfenol nanowires above or below a body whose internal structures are to be imaged; and
an RF, electrical, or a magnetic field source opposing said array such that the field effects are modulated by the body, and variations in the bodily structure will be manifested by variations in deflection of the nanowires.

15. The device of claim 14, wherein the deflections may be sensed using optical sensors viewing the tops of the nanowires.

16. The device of claim 14, wherein the deflections and the correlated changes in their current output may be sensed using giant magnetic resistance sensors operating near the base of the nanowires.

17. The device of claim 14, wherein the linear array is a grid matrix in two dimensions.

18. The device of claim 14, wherein the array may be stationary and the RF, electric or magnetic field source moves.

19. The device of claim 14, wherein the array moves and the RF, electric, or magnetic field source is stationary.

20. A device for vivo human and animal medical diagnostics and therapeutics, comprising:
Galfenol nanoparticles with embedded, attached chemotherapeutic compounds, comprising molecules, viruses, proteins, enzymes, cells, chemicals, whereby the nanoparticle is injected and subsequently controlled, monitored, tracked, and/or guided to a desired target point in a body for sensing, therapy, or to be triggered within the body by external radio-frequency (“RF”) or magnetic fields.

21. The device of claim 20, wherein Galfenol nanowires of varying diameter and length are used such that an external RF or magnetic field may induce heating of the nanowire, with the objective of thermal interaction with the targeted cell or body feature.

22. The device of claim 20, further comprising nanobots of structural shape such that the auxetic feature of the Galfenol causes a synchronous change in the dimensions and shape of the structure, resulting in a propulsion of the structure through fluids such as might be found throughout the body.

23. The device of claim 22, wherein the nanobot may take the form of a hollow-truncated cone, such that a magnetic stress in one direction will induce an expansion and volumetric change in a second dimension, inducing a swimming impulse, forcing the cone along the direction of the magnetic stress.

24. The device of claim 22, wherein pulsating radio-frequency emissions may be used to induce corresponding pulsations in the cone's diameter, resulting in a longitudinal vibration of the nanobot, with potential mechanical effects on the surrounding tissues and cells, to include the possible destruction of cell walls, selective resection, or ablation of cell constituents.

25. The device of claim 20, wherein the nanobots may be bar-coded, enabling remote readout of the location and effectiveness of a particular cargo, as transported on an individual nanobot.
